(19) United States
(12) Patent Application Publication
(10) Pub. No.: US 2024/0024621 A1
(43) Pub. Date: Jan. 25, 2024

(54) Introducer for an interventional device, particularly a delivery catheter for TAVI, comprising an expandable sheath

(71) Applicant: BIOTRONIK AG, Buelach (CH)

(72) Inventors: Lena Wiegmann, Zuerich (CH); Carina Haber, Singen (DE); Johannes Schampel, Birmensdorf (CH); Markus Hepke, Waltrop (DE)

(21) Appl. No.: 18/254,934

(22) PCT Filed: Dec. 7, 2021
(86) PCT No.: PCT/EP2021/084624

§ 371 Date: May 30, 2023

(30) Foreign Application Priority Data
Dec. 8, 2020 (DE) 202020107060.6

(51) Int. Cl. A61M 25/00; A61M 25/06

(52) U.S. Cl. CPC A61M 25/0023; A61M 25/0662; A61M 2025/0024; A61M 2025/1052

(57) ABSTRACT

A delivery system includes an interventional device having a distal end portion and an introducer configured to insert the interventional device into a vessel of a patient. The introducer has a tubular sheath configured to be inserted into the vessel defining a lumen to guide the interventional device into the vessel. The introducer includes a fastening mechanism configured to releasably fasten the introducer to the interventional device to prevent movement of the introducer with respect to the interventional device along a longitudinal axis.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2021/084624, which was filed Dec. 7, 2021, which application claimed priority from German Application Serial Number 20 2020 107 060.6, which was filed Dec. 8, 2020.

### FIELD OF THE INVENTION

A field of the invention is delivery systems that are configured for the vessel of a patient.

### BACKGROUND

Regarding such delivery systems, the insertion and withdrawal of the interventional device into/from a blood vessel with a critical inner diameter (inner vessel diameter near outer diameter of interventional device) generally bears a risk for complications.

Regarding introducing interventional devices into a vessel system of a patient, several approaches are known in the prior art.

In the so-called sheathless access, no introducer sheath is used. The outer diameter of the interventional device defines the critical blood vessel diameter into which it can be inserted. Hemostatic sealing is ensured by sutures of closure devices, which control the sealing of the vessel to the interventional device. The guiding of the interventional device through peripheral vessels is achieved by guidewire only.

An alternative approach corresponds to using an introducer in the form of an inline sheath. Here, the outer diameter of the interventional device defines the critical blood vessel diameter into which it can be inserted. An inline sheath is pre-mounted on the interventional device and ensures hemostatic sealing to the environment and additional guidance through the peripheral vessels as the interventional device can be moved relatively to the inline sheath. Particularly, the interventional device replaces the dilator of a common introducer.

Some introducers include an expandable sheath. Here, the expanded outer diameter of the introducer sheath defines the critical vessel diameter. The introducer sheath can expand and stretches the blood vessel radially. The expansion/contraction can be executed actively by accessories or passively by the interventional device itself. A dilator is needed for insertion into the peripheral vessel.

However, regarding sheathless accesses, the vessel access site is often stressed massively and must be observed continuously by the physician. Hemostatic sealing must be observed to and controlled by the operator manually resulting in a rather high risk of complications such as trauma, blood loss or rupture. Further, when using inline sheaths as described above where the withdrawal of interventional device can be realized with the introducer sheath only, a rather high risk of complications is probable in case of frequent and/or quick system retrieval (e.g. in case of an emergency or a post procedural intervention). Furthermore, an access via the expandable introducer sheath described above stretches the blood vessel and the access site during insertion and withdrawal which could lead to complications (e.g. ruptures) in combination with calcifications or incorrect vessel diameter determination.

### SUMMARY OF THE INVENTION

A preferred embodiment delivery system is configured for a vessel of a patient. The system includes an interventional device, and an introducer configured to introduce the interventional device into the vessel of the patient. The introducer includes a fastening mechanism configured to releasably fasten the introducer to the interventional device to prevent movement of the introducer with respect to the interventional device along a longitudinal axis (z). The fastening mechanism can be an inflatable balloon connected to a hub and configured to press against the interventional device when being inflated to fasten the introducer to the interventional device. The fastening mechanism can be a clamp connected to a hub, which clamp is configured to clamp the introducer to the interventional device to fasten the introducer to the interventional device. The fastening mechanism can be a first member connected to a hub and a separate second member configured to be releasably engaged with the first member, wherein the second member presses against the interventional device when being engaged with the first member, to fasten the introducer to the interventional device. The fastening mechanism can be at least one pin configured to be inserted into an opening of a proximal portion of the introducer to press against the interventional device and thereby fasten the introducer to the interventional device. The fastening mechanism can be an elongated flexible member guided by a proximal portion of the introducer and configured to tighten around the interventional device when being pulled on to fasten the introducer to the interventional device. The fastening mechanism can be a tensioning chain having a plurality of chain links, wherein at least one chain link is connected to a proximal portion of the introducer, and wherein the tensioning chain is configured to be tightened around the interventional device to fasten the introducer to the interventional device. The fastening mechanism can be a spring element connected to a proximal portion of the introducer and configured to be pretensioned against the interventional device to fasten the introducer to the interventional device. The fastening mechanism can be a stopping element comprising a longitudinal gap to allow arranging the stopping element on the interventional device so that the stopping element extends between a proximal end of the introducer and a distal end of a handle of the interventional device so that the introducer is fastened to the interventional device through the stopping element. The fastening mechanism can be a screw configured to be screwed into an inner thread of an opening of a proximal portion of the introducer to press against the interventional device and thereby fasten the introducer to the interventional device. The fastening mechanism can be an electromagnet an electrical coil wound around a metal core, wherein the electromagnet is arranged on a proximal portion of the introducer and is configured to attract a metal sheath of the interventional device when an electrical current is applied to the electrical coil to fasten the introducer to the interventional device.

### BRIEF DESCRIPTION OF THE DRAWINGS

In the following further features and advantages of the present invention shall be described with reference to the Figures which show preferred embodiments of the present invention, wherein

FIG. 1 shows an embodiment of a delivery system according to the present invention during three different stages, wherein the upper part of FIG. 1 shows insertion of the interventional device with the pre-mounted introducer thereon into a vessel of the patient using a distal portion of the interventional device as a dilator, and wherein the middle part of FIG. 1 shows removal of the interventional device through a sheath of the introducer which is capable of expanding to let the interventional device pass through the sheath, and wherein the lower part of FIG. 1 shows the introducer's sheath remaining in the vessel, wherein a hemostatic valve of the hub of the introducer seals the lumen of the introducer;

FIG. 2 shows schematical illustration of a locking mechanism of a sheath of the introducer that allows to prevent expansion of the sheath of the introducer;

FIG. 3 shows the locking mechanism in a released state that allows the sheath of the introducer to expand; and

FIGS. 4 to 19 show different embodiments of a fastening mechanism for fastening the introducer to the interventional device.

### DETAILED DESCRIPTION OF THE PREFERRED EMBODIMENTS

A preferred embodiment provides a delivery system that includes an introducer and an interventional device that allow reducing stress on the access site and peripheral vessels, wherein particularly the critical diameter shall be approximated to the outer diameter of the interventional device.

A preferred delivery system for introducing an interventional device into a vessel of a patient includes an interventional device having a distal end portion, and an introducer for inserting the interventional device into a vessel of the patient, wherein the introducer includes a tubular sheath extending along a longitudinal axis, wherein the sheath is configured to be inserted into the vessel and surrounds a lumen for guiding the interventional device into the vessel, wherein the introducer is pre-mounted on the interventional device such that the interventional device extends through the lumen of the sheath of the introducer and protrudes out of the lumen with the distal end portion of the interventional device, wherein the distal end portion of the interventional device forms a dilator for inserting the sheath of the introducer into the vessel, and wherein the sheath of the introducer is configured to be expanded from a first state to a second state, wherein an inner diameter of the sheath in the first state is smaller than an inner diameter of the sheath in the second state.

Proximal” as used herein means that a corresponding portion or component of the system is closer to the physician or handle of the interventional device than a corresponding “distal” portion or component of the system, which is further away from the physician/handle along the longitudinal axis.

According to a preferred embodiment of the present invention, the introducer includes a hub connected to a proximal end of the sheath, the hub including an opening for inserting the interventional device into the lumen of the sheath or for removing the interventional device from the lumen of the sheath of the introducer, wherein the hub includes a hemostatic valve for sealing the opening when the interventional device is removed from the sheath of the introducer.

Furthermore, according to an embodiment of the present invention, in the second state of the sheath of the introducer, the distal end portion of the interventional device is allowed to pass through the lumen of the sheath upon removal of the interventional device from the introducer.

Furthermore, according to an embodiment of the present invention, the introducer includes a locking mechanism configured to maintain the sheath in the first state, wherein the locking mechanism is releasable to allow the sheath to be expanded into the second state, particularly upon passage of the distal end portion of the interventional device through the lumen of the sheath.

Furthermore, according to an embodiment of the present invention, the locking mechanism includes a ring member extending along a periphery of the sheath.

Preferably, in an embodiment, the ring member includes two ends configured to be connected to one another to maintain the sheath in the first state, wherein the introducer includes an elongated pulling member connected to the ring member such that when the pulling member is pulled in the proximal direction, the two ends of the ring member are disconnected and the sheath is allowed to expand to the second state.

According to yet another embodiment, the introducer includes a fastening mechanism configured to releasably fasten the introducer to the interventional device to prevent movement of the introducer with respect to the interventional device along the longitudinal axis.

Furthermore, according to a preferred embodiment of the present invention, the interventional device is a delivery catheter for delivering an implant to an implantation site. For example, the delivery catheter is configured for TAVI, i.e. the implant is a self-expandable prosthetic aortic heart valve. Furthermore, the distal end portion of the interventional device/delivery catheter includes a slidable capsule for carrying the implant (e.g. the self-expandable prosthetic aortic heart valve).

Furthermore, the sheath can be elastically expandable from the first state to the second state and/or may include portions that are configured to be movable, e.g. deployable, to allow expansion from the first state to the second state. For instance, the sheath can include e.g. one or two sections extending along the longitudinal axis and including overlapping wall portions or a corrugated wall portion, respectively.

According to an embodiment, the fastening mechanism includes a slider configured to be slid into a gap between the interventional device and a proximal portion of the introducer so as to fasten the introducer to the interventional device. Particularly, the slider can be a wedge. The proximal portion of the introducer can be formed by the hub.

According to a further embodiment, the fastening mechanism includes an inflatable balloon connected to the hub and configured to press against the interventional device when being inflated to fasten the introducer to the interventional device.

According to a further embodiment, the fastening mechanism includes a clamp connected to the hub, which clamp is configured to clamp the introducer to the interventional device to fasten the introducer to the interventional device.

According to a further embodiment, the fastening mechanism includes a first member being connected to the hub and a separate second member configured to be releasably engaged with the first member, wherein the second member presses against the interventional device when being engaged with the first member, to fasten the introducer to the interventional device. For engaging with the first member, the second member can include a plurality of fingers that are configured to engage with a plurality of recesses of the first member. Furthermore, the first and the second member can be configured to assist the engagement of the second member with the first member by a magnetic force. For this at least one of the two members can include a permanent magnet.

According to a further embodiment, the fastening mechanism includes at least one pin being configured to be inserted into an opening of a proximal portion of the introducer so as to press against the interventional device and thereby fasten the introducer to the interventional device.

According to a further embodiment, the fastening mechanism includes an elongated flexible member (e.g. a thread or rope or wire) guided by a proximal portion of the introducer and configured to tighten around the interventional device when being pulled on to fasten the introducer to the interventional device.

According to a further embodiment, the fastening mechanism includes a tensioning chain including a plurality of chain links, wherein at least one chain link is connected to a proximal portion of the introducer, and wherein the tensioning chain is configured to be tightened around the interventional device to fasten the introducer to the interventional device.

According to a further embodiment, the fastening mechanism includes a spring element connected to a proximal portion of the introducer and configured to be pretensioned against the interventional device to fasten the introducer to the interventional device.

According to a further embodiment, the fastening mechanism includes a stopping element including a longitudinal gap to allow arranging the stopping element on the interventional device so that the stopping element extends between a proximal end of the introducer and a distal end of a handle of the interventional device so that the introducer is fastened to the interventional device through the stopping element.

According to a further embodiment, the fastening mechanism includes a screw being configured to be screwed into an inner thread of an opening of a proximal portion of the introducer to press against the interventional device and thereby fasten the introducer to the interventional device.

According to a further embodiment, the fastening mechanism includes an electromagnet including an electrical coil wound around a metal core, wherein the electromagnet is arranged on a proximal portion of the introducer and is configured to attract a metal sheath of the interventional device when an electrical current is applied to the electrical coil to fasten the introducer to the interventional device. Particularly the metal core is an L-shaped metal core including a face side facing the metal sheath/interventional device.

According to a further embodiment, the delivery system includes an electronic control unit configured to control the respective fastening mechanism.

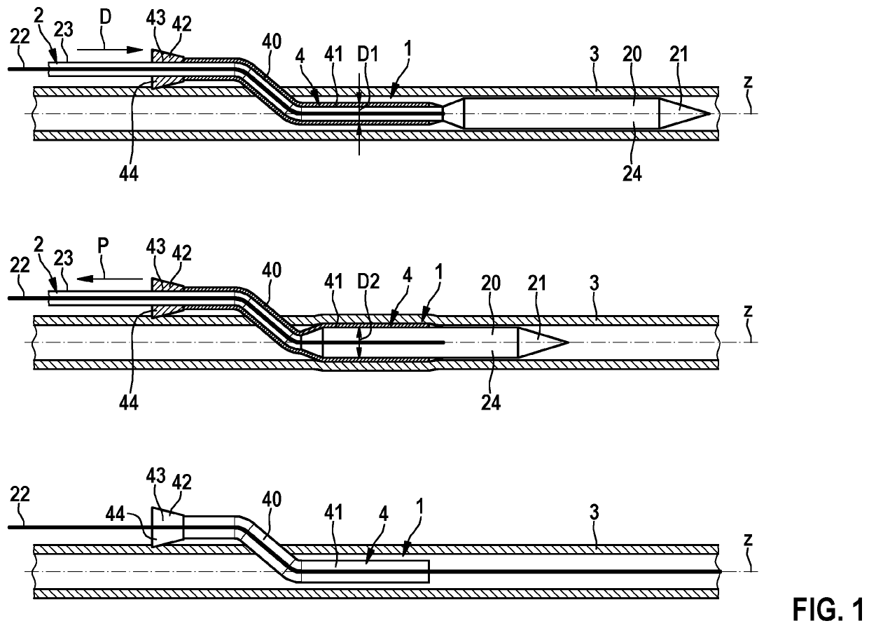

FIG. 1 shows an embodiment of a delivery system 1 according to the present invention, wherein the delivery system 1 includes an interventional device 2 including a distal end portion 20. Particularly, the interventional device 2 can be a delivery catheter for delivering an implant (not shown) to an implantation site, wherein the distal end portion 20 of the interventional device 2 includes a capsule 24 for carrying the implant. Particularly, the delivery catheter 2 is configured for TAVI (transcatheter aortic valve implantation), i.e. the implant is an (e.g. self-expandable) prosthetic aortic heart valve. The delivery catheter 1 can further include an outer sheath 23 connected to the capsule 24. The capsule 24 can be slidable with respect to an inner sheath (not shown) of the delivery catheter, to which inner sheath the implant is connected before being released by removing the capsule 24 from the implant in the proximal direction P. The delivery catheter further includes a guidewire lumen for receiving a guidewire 22 that can exit the delivery catheter at a distal catheter tip 21 of the delivery catheter. Using the guidewire 22, the interventional device/delivery catheter can be guided to the implantation site after insertion of the interventional device 2 into the vessel 3 of a patient through an incision of the vessel 3.

The delivery system 1 further includes an introducer 4 being pre-mounted on the interventional device 2 as shown in the upper part of FIG. 1. The introducer 4 includes a tubular sheath 40 extending along a longitudinal axis z, wherein the sheath 40 surrounds a lumen 41 for guiding the interventional device 2 into the vessel 3.

Furthermore, the introducer 4 includes a hub 42 connected to a proximal end of the sheath of the hub 42, wherein the hub 42 includes an opening 43 for inserting the interventional device 2 into the lumen 41 of the sheath 40 or for removing the interventional device 2 from the lumen 41 of the sheath 40 of the introducer 4. The hub 42 further includes a hemostatic valve 44 for sealing the opening 43 of the hub 42 when the interventional device 2 is removed from the sheath 40 of the introducer 4 (cf. lower part of FIG. 1).

When introducing the interventional device 2 together with the introducer 4 pre-mounted thereon into the vessel 3 in the distal direction D as shown in the upper part of FIG. 1, the distal portion 20 of the interventional device 2 protrudes out of the introducer 4 and functions as a dilator.

Furthermore, according to the present invention, the sheath 40 of the introducer 4 is configured to be expanded from a first state to a second state, wherein an inner diameter D1 of the sheath 40 in the first state (upper part of FIG. 1) is smaller than an inner diameter D2 of the sheath 40 in the second state (middle part of FIG. 1).

Particularly, the fact that the sheath 40 can be expanded to assume its second expanded state allows the distal end portion 20 of the interventional device 2 to pass through the lumen 41 of the sheath 40 upon removal of the interventional device 2 from the vessel 3 as shown in the middle part of FIG. 1. The lower part of FIG. 1 shows the introducer 4 remaining in the vessel 3 after complete removal of the interventional device 2 from the introducer 4/vessel 3.

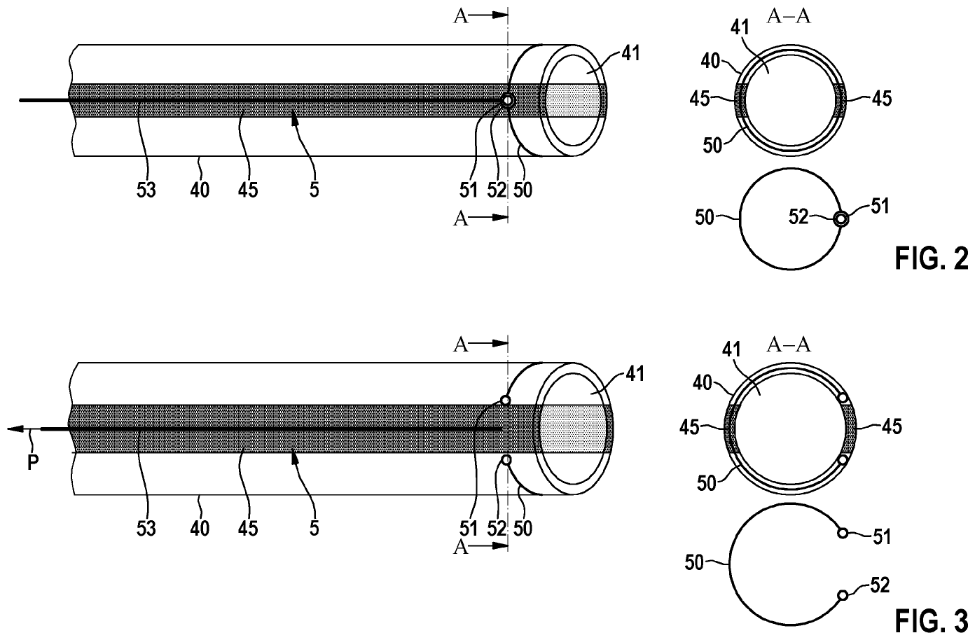

Particularly, to control expansion or expandability of the sheath 40 of the introducer 4, the introducer 4 can include a locking mechanism 5 as shown in FIGS. 2 and 3 configured to maintain the sheath 40 in the first state as shown in FIG. 2, wherein the locking mechanism 5 is releasable to allow the sheath 40 to be expanded into the second state as shown in FIG. 3.

Particularly, the locking mechanism 5 can include a ring member 50 extending along a periphery of the sheath 40 of the introducer 4, wherein the ring member 50 can include two end portions 51, 52 configured to be connected to one another to maintain the sheath 40 in the first state (cf. FIG. 2), wherein the introducer 4 includes an elongated pulling member 53 connected to the ring member 50 such that when the pulling member 53 is pulled in the proximal direction P, the two end portions 51, 52 of the ring member 50 are disconnected from one another, the ring member 50 is allowed to open up and expand radially and the sheath 40 is thereby allowed to expand radially to the second state (cf. FIG. 3). The introducer sheath 40 surrounds a lumen 41. The sheath 40 of the introducer 4 is configured to be expanded from a first state to a second state.

As already described above, the expansion of the tubular sheath 40 of the introducer 4 can be realized in a variety of fashions. For example, the sheath 4 can include two opposing longitudinal sections 45 extending along the longitudinal axis z, wherein these sections 45 can e.g. include overlapping wall portions or a corrugated wall portion that allow the sheath 40 to assume a larger diameter D2 when the constraint set by the ring member 50 is removed by disconnecting the end portions 51, 52 of the ring member 50 from one another.

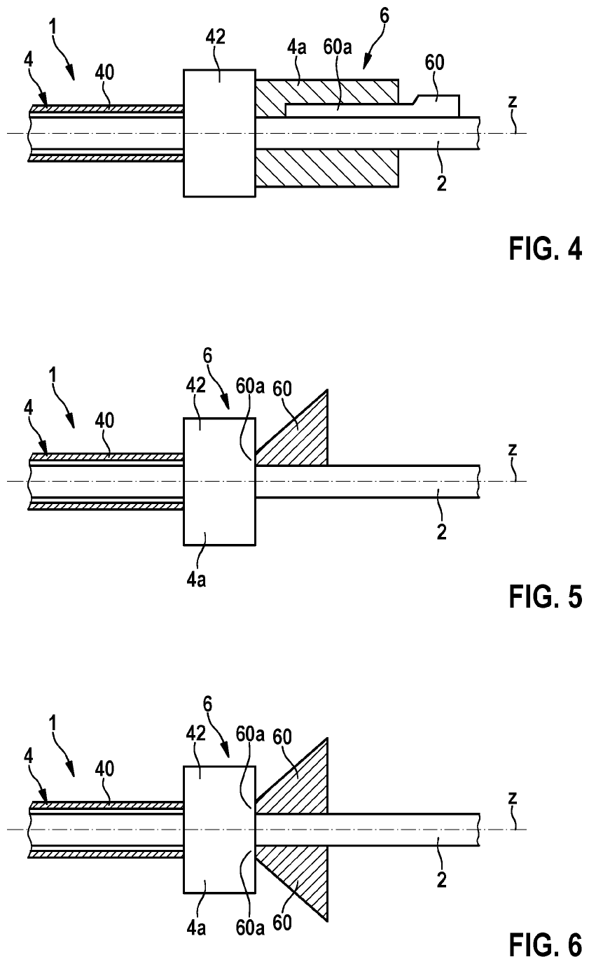

As shown in FIGS. 4 to 19, the introducer 4 of the delivery system 1 can further include a fastening mechanism 6 configured to releasably fasten the introducer 4 to the interventional device 2 so as to prevent movement of the introducer 4 with respect to the interventional device 2 along the longitudinal axis z. The different fastening mechanisms shown in FIGS. 4 to 19 will be explained in detail below.

Particularly, according to an embodiment shown in FIG. 4, the fastening mechanism 6 includes a slider 60 configured to be slid into a gap 60a between the interventional device 2 and a proximal portion 4a of the introducer 4 so as to fasten the introducer 4 to the interventional device 2. The proximal portion 4a can be connected to the hub 42 of the introducer or can be an integral part of the hub 42. The sheath 40 of the introducer 4 is configured to be expanded from a first state to a second state. Briefly, FIG. 4 shows a delivery system 1 with a fastening mechanism 6 wherein proximal to the hub 42 of the introducer 4 the slider 60 may be firmly connected to the hub 42. The introducer 4 is attached to the catheter by pushing the slider 60 in, so that it cannot be moved either in the distal or proximal direction. By pulling the slider 60 out, the introducer 4 can be moved.

Particularly, as shown in FIG. 5, the slider 60 can be a wedge, wherein the proximal portion 4a of the introducer 4 that forms the gap 60a with the interventional device 2 that receives the wedge can be formed by the hub 42. Particularly, as shown in FIG. 6, more than one wedge, i.e. two wedges, can be used for fastening the introducer 4 to the interventional device 2. The sheath 40 of the introducer 4 is configured to be expanded from a first state to a second state. Briefly, FIGS. 5 and 6 each show a delivery system 1 with a fastening mechanism 6 wherein one or more wedges can be inserted into the hub 42 or into a part proximal of the hub 42. As a result, the introducer 4 can no longer be moved.

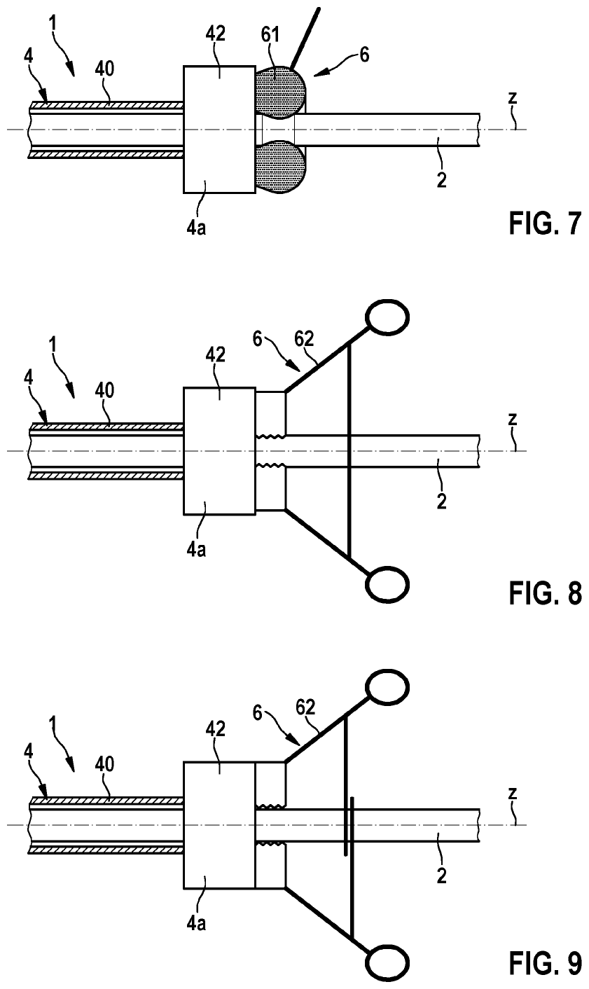

Furthermore, according to the embodiment shown in FIG. 7, the fastening mechanism 6 includes an inflatable balloon 61 connected to the hub 42 and configured to press against the interventional device 2 when being inflated so as to fasten the introducer 4 to the interventional device 2. The sheath 40 of the introducer 4 is configured to be expanded from a first state to a second state. The introducer 4 has proximal portion 4a which is an integral part of the hub 42. Briefly, FIG. 7 shows a delivery system 1 with a fastening mechanism 6 wherein proximal to the hub 42 the inflatable and deflatable balloon 61 is connected to the hub 42. When inflating the balloon 61 the introducer is fixed on the catheter. When the balloon is deflated, the introducer 4 can be moved.

FIGS. 8 and 9 show a further embodiment of the fastening mechanism 6, wherein here the fastening mechanism 6 includes a clamp 62 connected to the hub 42 of the introducer 4, wherein the clamp 62 is configured to clamp the introducer 4 to the interventional device 2 in a closed position of the clamp 62 so as to fasten the introducer 4 to the interventional device 2 (cf. FIG. 8), and wherein the clamp 62 releases the interventional device 2 in an open position of the clamp 62 as shown in FIG. 9 allowing movement of the interventional device 2 along the longitudinal axis z with respect to the introducer 4. The sheath 40 of the introducer 4 is configured to be expanded from a first state to a second state. The introducer 4 has proximal portion 4a which is an integral part of the hub 42. Briefly, FIGS. 8 and 9 each show a delivery system 1 with a fastening mechanism 6 wherein the proximal part of the hub 42 is connected with the clamp 62. When the clamp 62 is closed the introducer 42 is fixed. The introducer 42 can be moved when the clamp is open (fixation like surgical scissors).

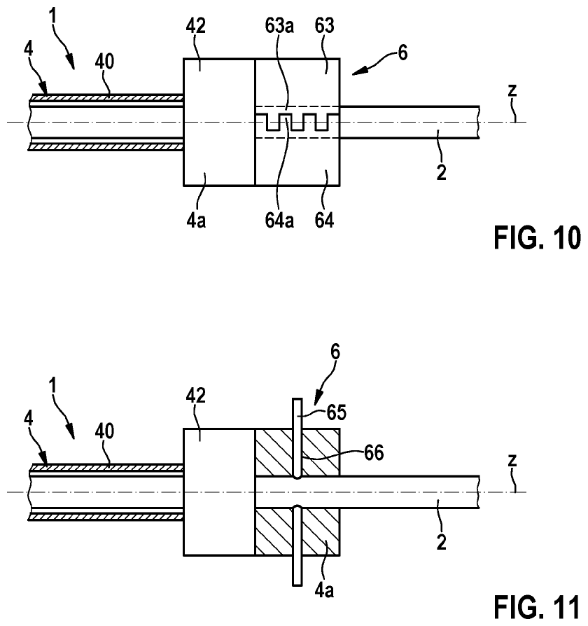

According to a further embodiment shown in FIG. 10, the fastening mechanism 6 includes a first member 63 being connected to the hub 42 and a separate second member 64 configured to be releasably engaged with the first member 63, wherein the second member 64 presses against the interventional device 2 when being engaged with the first member 63, so as to fasten the introducer 4 to the interventional device 2. For engaging with the first member 63, the second member 64 can include a plurality of fingers 64a that are configured to engage with a plurality of recesses 63a of the first member 63. Furthermore, the first and the second member 63, 64 can be configured to assist the engagement of the second member 64 with the first member 63 by a magnetic force. For this, at least one of the two members 63, 64 can include a permanent magnet. The sheath 40 of the introducer 4 is configured to be expanded from a first state to a second state. The introducer 4 has proximal portion 4a which is an integral part of the hub 42. Briefly, FIG. 10 shows a delivery system 1 with a fastening mechanism 6 wherein the proximal end of the hub 42 is connected with one of the two members of the fastening mechanism 6. The second member 64 can be removed. Both members 63, 64 have fingers which fit into each other and a soft elastic part inside. By joining the fingers together the elastic part is compressed so much that the introducer 42 on the catheter cannot be moved anymore.

Furthermore, according to the embodiment shown in FIG. 11, the fastening mechanism 6 includes at least one pin 65 being configured to be inserted into an opening 66 of a proximal portion 4a of the introducer 4 so as to press against the interventional device 2 and to thereby fasten the introducer 4 to the interventional device 2. Particularly, the proximal portion 4a can be connected to the hub 42 of the introducer 4 or may form an integral part of the hub 42. The sheath 40 of the introducer 4 is configured to be expanded from a first state to a second state. The proximal portion 4a is connected to the hub 42 of the introducer 4. Briefly, FIG. 11 shows a delivery system 1 with a fastening mechanism 6 wherein one or more pins 65 in a part fixed on the proximal region of the hub 42 can be sticked in to fix the introducer 4 on the catheter.

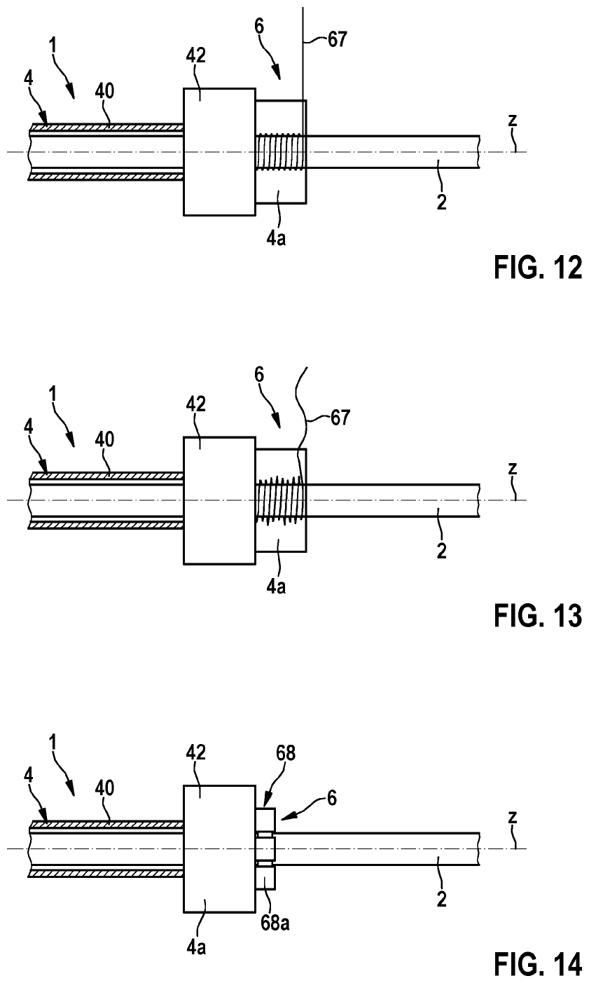

Further, according to the embodiment shown in FIGS. 12 and 13, the fastening mechanism 6 includes an elongated flexible member 67 (e.g. a thread 67) guided by a proximal portion 4a of the introducer 4, wherein the flexible member 67 is configured to tighten around the interventional device 2 when being pulled on to fasten the introducer 4 to the interventional device 2 (cf. FIG. 12). Particularly, the proximal portion 4a can be connected to the hub 42 or can form in integral part of the hub 42 of the introducer 4. The sheath 40 of the introducer 4 is configured to be expanded from a first state to a second state. The proximal portion 4a is connected to the hub 42 of the introducer 4. Briefly, FIGS. 12 and 13 each show a delivery system 1 with a fastening mechanism 6 wherein a thread or string may be in a part proximal of the hub 42. The hub 42 and the part are connected together. When the string is pulled, the lacing becomes tighter and the introducer 4 is fixed.

Furthermore, according to the embodiment shown in FIG. 14, the fastening mechanism 6 includes a tensioning chain 68 including a plurality of chain links 68a, wherein at least one chain link 68a is connected to a proximal portion 4a of the introducer 4. The proximal portion 4a of the introducer 4 can be formed by the hub 42 of the introducer 4. Particularly, the tensioning chain 68 is configured to be tightened around the interventional device 2 so as to fasten the introducer 4 to the interventional device 2. The sheath 40 of the introducer 4 is configured to be expanded from a first state to a second state. The introducer 4 has proximal portion 4a which is an integral part of the hub 42. Briefly, FIG. 14 shows a delivery system 1 with a fastening mechanism 6 wherein one or more parts of the tensioning chain 68 is connected to the proximal part of the hub 42. If the tensioning chain 68 is closed, the introducer 4 can no longer be moved back and forth on the catheter.

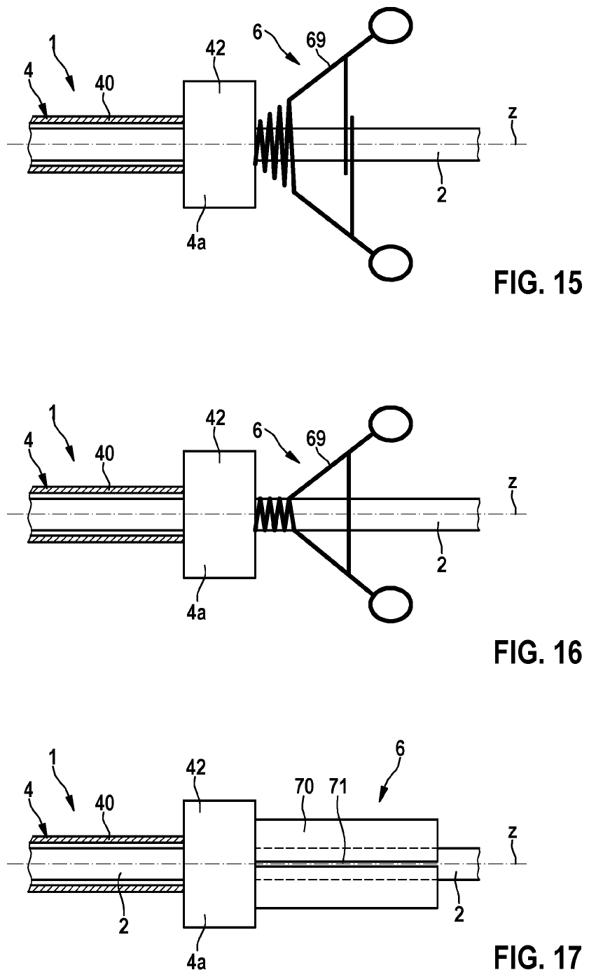

FIGS. 15 and 16 show yet another embodiment of the fastening mechanism 6 of the introducer 4. Here, the fastening mechanism 6 includes a spring element 69 connected to a proximal portion 4a (e.g. hub 42) of the introducer 4 and configured to be pretensioned against the interventional device 2 so as to fasten the introducer 4 to the interventional device 2. FIG. 15 shows the spring element 69 being tensioned against the interventional device 2 so that the introducer 4 is fastened to the interventional device 2, whereas FIG. 16 shows the spring element 69 being deformed in a manner so as to release the interventional device 2. The sheath 40 of the introducer 4 is configured to be expanded from a first state to a second state. The introducer 4 has proximal portion 4a which is an integral part of the hub 42. Briefly, FIGS. 15 and 16 each show a delivery system 1 with a fastening mechanism 6 wherein by closing the spring element 69 (a tensioning feather), which is connected to the distal part of the hub 42, the introducer 4 is fixed on the catheter.

Further, according to the embodiment shown in FIG. 17, the fastening mechanism 6 of the introducer includes a stopping element 70 including a longitudinal gap 71 to allow arranging the stopping element 70 on the interventional device 2 so that the stopping element 70 extends between a proximal end 4a of the introducer 4 and a distal end of a handle of the interventional device 2, which allows fastening the introducer 4 to the interventional device 2 through the stopping element 70. The sheath 40 of the introducer 4 is configured to be expanded from a first state to a second state. The introducer 4 has proximal portion 4a which is an integral part of the hub 42. Briefly, FIG. 17 shows a delivery system 1 with a fastening mechanism 6 wherein the stopping element 70 with a longitudinal cut is between the proximal end of the hub 42 and the distal part of the handle of the introducer 4. Via the stopping element 70 the introducer 4 is fixed.

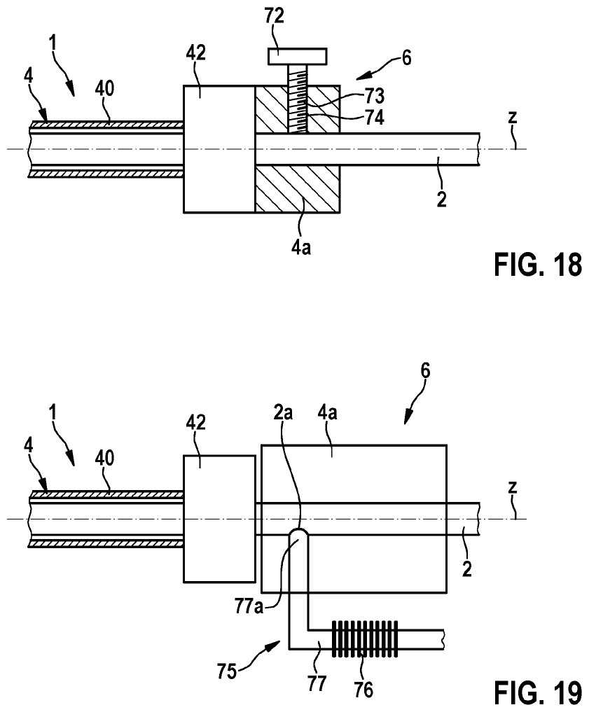

According to a further embodiment of the fastening mechanism 6 shown in FIG. 18, the fastening mechanism 6 includes a screw 72 being configured to be screwed into an inner thread 73 of an opening 74 of a proximal portion 4a of the introducer 4 so as to press against the interventional device 2 and thereby fasten the introducer 4 to the interventional device 2. Particularly, the proximal portion 4a can be connected to the hub 42 or may form an integral part of the hub 42. The sheath 40 of the introducer 4 is configured to be expanded from a first state to a second state. The proximal portion 4a is connected to the hub 42 of the introducer 4. Briefly, FIG. 18 shows a delivery system 1 with a fastening mechanism 6 wherein a screw 72 in a part connected to the proximal part of the hub 42 can be screwed in and out. When the screw 72 is screwed in, the introducer 4 is fixed on the catheter.

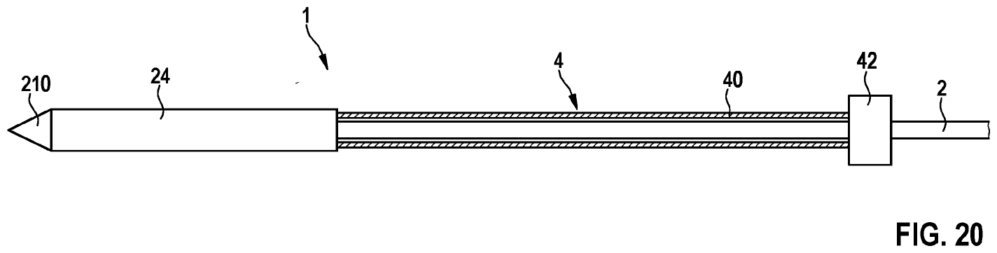

Finally, according to yet another embodiment shown in FIG. 19, the fastening mechanism 6 includes an electromagnet 75 including an electrical coil 76 wound around a metal core 77, wherein the electromagnet 75 is arranged on a proximal portion 4a of the introducer 4 and is configured to attract a metal sheath or member 2a of the interventional device 2 when an electrical current is applied to the electrical coil 76 to fasten the introducer 4 to the interventional device 2 by attracting the metal sheath/member 2a. Particularly, the metal core 77 is an L-shaped metal core 77 including a face side 77a facing the metal sheath 2a. The sheath 40 of the introducer 4 is configured to be expanded from a first state to a second state. The proximal portion 4a is connected to the hub 42 of the introducer 4. This fastening mechanism 6 in FIG. 19 can be seen as a braking device on the introducer 4 which will hold or release the parallel metal shaft from the catheter. This braking device (coil and core) is fixed to the proximal end of the introducer 4. A cylindrical coil 76 is a coil in which the wire winding lies on a cylindrical jacket. The cylindrical coil with an L-shaped metal core 77, which is fixed on the introducer 4, is magnetized by a battery current. The user has an ON/OFF button at his disposal, wherein ON means current flows thus braking occurs and OFF means no current flows and thus no magnetic field/magnetic force is induced. Thus, a relative movement between the catheter and the introducer 4 is possible.

FIG. 20 shows a schematic drawing of the delivery system 1 including an interventional device 2 having a distal tip 210, preferably a catheter having a distal catheter tip, and an introducer 4 for inserting the interventional device 2 into a vessel of the patient. The introducer 4 includes an introducer hub 42. The distal end portion of the interventional device 2 includes a slidable capsule 24 for carrying the implant. The sheath 40 of the introducer 4 is configured to be expanded from a first state to a second state, wherein an inner diameter of the sheath 40 in the first state is smaller than an inner diameter D2 of the sheath 40 in the second state.

Preferably, the introducer 4 includes a tubular sheath extending along a longitudinal axis, wherein the sheath is configured to be inserted into a vessel of a patient and surrounds a lumen for guiding the interventional device into the vessel.

The introducer 4 can be pre-mounted on the interventional device 2 such that the interventional device 2 extends through the lumen of the sheath 40 and protrudes out of the lumen 41 with the distal end portion of the interventional device 2. The distal end portion forms a dilator for inserting the sheath of the introducer 4 into the vessel 3 of a patient.

Such a delivery system of FIG. 20 can include one of the fastening mechanisms as described in FIGS. 4 to 19.

The present invention enables the user of the delivery system in an advantageous manner to put less stress to the access site during insertion of the introducer and interventional device compared to a sheathless entry or a stand-alone expandable sheath, as the access and inner diameter of the vessel are less stretched. Only in case of an emergency or post procedural intervention when the interventional device is withdrawn through the introducer, the vessel and access site are stressed. After withdrawal, a regular access is still present through the introducer remaining in the peripheral vessel of the patient to allow further interventional devices to pass through. The critical vessel diameter is reduced to the outer diameter of the interventional device if no withdrawal through the expandable inline sheath is performed.

1-13. (canceled)

14. A delivery system configured for a vessel of a patient, comprising:
an interventional device, and
an introducer configured to introduce the interventional device into the vessel of the patient, wherein the introducer comprises a fastening mechanism configured to releasably fasten the introducer to the interventional device to prevent movement of the introducer with respect to the interventional device along a longitudinal axis (z),
wherein the fastening mechanism comprises an inflatable balloon connected to a hub and configured to press against the interventional device when being inflated to fasten the introducer to the interventional device, or
the fastening mechanism comprises a clamp connected to a hub, which clamp is configured to clamp the introducer to the interventional device to fasten the introducer to the interventional device, or
the fastening mechanism comprises a first member connected to a hub and a separate second member configured to be releasably engaged with the first member, wherein the second member presses against the interventional device when being engaged with the first member, to fasten the introducer to the interventional device, or
the fastening mechanism comprises at least one pin configured to be inserted into an opening of a proximal portion of the introducer to press against the interventional device and thereby fasten the introducer to the interventional device, or
the fastening mechanism comprises an elongated flexible member guided by a proximal portion of the introducer and configured to tighten around the interventional device when being pulled on to fasten the introducer to the interventional device, or
the fastening mechanism comprises a tensioning chain comprising a plurality of chain links, wherein at least one chain link is connected to a proximal portion of the introducer, and wherein the tensioning chain is configured to be tightened around the interventional device to fasten the introducer to the interventional device, or
the fastening mechanism comprises a spring element connected to a proximal portion of the introducer and configured to be pretensioned against the interventional device to fasten the introducer to the interventional device, or
the fastening mechanism comprises a stopping element comprising a longitudinal gap to allow arranging the stopping element on the interventional device so that the stopping element extends between a proximal end of the introducer and a distal end of a handle of the interventional device so that the introducer is fastened to the interventional device through the stopping element, or
the fastening mechanism comprises a screw configured to be screwed into an inner thread of an opening of a proximal portion of the introducer to press against the interventional device and thereby fasten the introducer to the interventional device, or
the fastening mechanism comprises an electromagnet comprising an electrical coil wound around a metal core, wherein the electromagnet is arranged on a proximal portion of the introducer and is configured to attract a metal sheath of the interventional device when an electrical current is applied to the electrical coil to fasten the introducer to the interventional device.

15. A delivery system configured for a vessel of a patient, comprising:
an interventional device comprising a distal end portion, and
an introducer configured to insert the interventional device into the vessel of the patient, wherein the introducer comprises a tubular sheath extending along a longitudinal axis (z), wherein the sheath surrounds a lumen configured to guide the interventional device into the vessel, wherein the interventional device extends through the lumen of the sheath, wherein said distal end portion forms a dilator configured to insert the sheath of the introducer into the vessel, and wherein the sheath of the introducer is configured to be expanded from a first state to a second state, wherein an inner diameter (D1) of the sheath in the first state is smaller than an inner diameter (D2) of the sheath in the second state, wherein the introducer comprises a fastening mechanism configured to releasably fasten the introducer to the interventional device to prevent movement of the introducer with respect to the interventional device along the longitudinal axis (z), and
wherein the fastening mechanism comprises an inflatable balloon connected to a hub and configured to press against the interventional device when being inflated to fasten the introducer to the interventional device, or
the fastening mechanism comprises a clamp connected to a hub, which clamp is configured to clamp the introducer to the interventional device to fasten the introducer to the interventional device, or
the fastening mechanism comprises a first member being connected to a hub and a separate second member configured to be releasably engaged with the first member, wherein the second member presses against the interventional device when being engaged with the first member, to fasten the introducer to the interventional device, or
the fastening mechanism comprises at least one pin being configured to be inserted into an opening of a proximal portion of the introducer to press against the interventional device and thereby fasten the introducer to the interventional device, or
the fastening mechanism comprises an elongated flexible member guided by a proximal portion of the introducer and configured to tighten around the interventional device when being pulled on to fasten the introducer to the interventional device, or
the fastening mechanism comprises a tensioning chain comprising a plurality of chain links, wherein at least one chain link is connected to a proximal portion of the introducer, and wherein the tensioning chain is configured to be tightened around the interventional device to fasten the introducer to the interventional device, or
the fastening mechanism comprises a spring element connected to a proximal portion of the introducer and configured to be pretensioned against the interventional device to fasten the introducer to the interventional device, or
the fastening mechanism comprises a stopping element comprising a longitudinal gap to allow arranging the stopping element on the interventional device so that the stopping element extends between a proximal end of the introducer and a distal end of a handle of the interventional device so that the introducer is fastened to the interventional device through the stopping element, or
the fastening mechanism comprises a screw being configured to be screwed into an inner thread of an opening of a proximal portion of the introducer to press against the interventional device and thereby fasten the introducer to the interventional device, or
the fastening mechanism comprises an electromagnet comprising an electrical coil wound around a metal core, wherein the electromagnet is arranged on a proximal portion of the introducer and is configured to attract a metal sheath of the interventional device when an electrical current is applied to the electrical coil to fasten the introducer to the interventional device.

16. The delivery system according to claim 15, wherein the introducer is pre-mounted on the interventional device such that the interventional device extends through the lumen of the sheath and protrudes out of the lumen with said distal end portion of the interventional device.

17. The delivery system according to claim 15, wherein the introducer comprises the hub connected to a proximal end of the sheath, wherein the hub comprises an opening configured to insert the interventional device into the lumen of the sheath or to remove the interventional device from the lumen of the sheath of the introducer, and wherein the hub comprises a hemostatic valve configured to seal the opening when the interventional device is removed from the sheath of the introducer.

18. The delivery system according to claim 15, wherein in the second state of the sheath, the distal end portion of the interventional device is allowed to pass through the lumen of the sheath upon removal of the interventional device from the sheath of the introducer.

19. The delivery system according to claim 15, wherein the interventional device is a delivery catheter configured to deliver an implant to an implantation site, wherein the distal end portion of the interventional device comprises a slidable capsule configured to carry the implant.

20. A delivery system configured for a vessel of a patient, comprising:
an interventional device comprising a distal end portion, and
an introducer configured to introduce the interventional device into a vessel of the patient, wherein the introducer comprises a tubular sheath extending along a longitudinal axis (z), wherein the sheath is configured to be inserted into the vessel and surrounds a lumen configured to guide the interventional device into the vessel, wherein the interventional device extends through the lumen of the sheath, wherein said distal end portion forms a dilator configured to insert the sheath of the introducer into the vessel, and wherein the introducer comprises a hub connected to a proximal end of the sheath, wherein the hub comprises an opening configured to insert the interventional device into the lumen of the sheath or to remove the interventional device from the lumen of the sheath of the introducer, wherein the hub comprises a hemostatic valve configured to seal the opening when the interventional device is removed from the sheath of the introducer.
